(19) United States
(12) Patent Application Publication
(10) Pub. No.: US 2019/0172569 A1
(43) Pub. Date: Jun. 6, 2019

(54) Presenting Contextually Relevant Patient Data to a Medical Professional

(71) Applicant: International Business Machines Corporation, Armonk, NY (US)

(72) Inventors: Jacob Green, Somerville, MA (US); Nicole Seo, San Francisco, CA (US); Tim C. Short, Somerville, MA (US)

(21) Appl. No.: 16/197,513

(22) Filed: Nov. 21, 2018

Related U.S. Application Data
(63) Continuation of application No. 15/829,362, filed on Dec. 1, 2017.

(51) Int. Cl. G16H 10/60; G16H 40/63

(52) U.S. Cl. CPC G16H 10/60; G16H 40/63

(57) ABSTRACT

A mechanism is provided for presenting contextually relevant patient data to a medical professional. Electronic medical records (EMRs) of a patient are analyzed to identify a medical condition associated with the patient. A set of treatments are identified from a corpus of medical treatment guidelines for the patient's medical condition. For each treatment in the set of treatments, a subset of treatments are identified that the patient has followed. The set of treatments for the patient's medical condition that the patient has followed are them presented to the medical professional.

### BACKGROUND

The present application relates generally to an improved data processing apparatus and method and more specifically to mechanisms for presenting contextually relevant patient data to a medical professional.

Decision-support systems exist in many different industries where human experts require assistance in retrieving and analyzing information. An example that will be used throughout this application is a diagnosis system employed in the healthcare industry. Diagnosis systems can be classified into systems that use structured knowledge, systems that use unstructured knowledge, and systems that use clinical decision formulas, rules, trees, or algorithms. The earliest diagnosis systems used structured knowledge or classical, manually constructed knowledge bases. The Internist-I system developed in the 1970s uses disease-finding relations and disease-disease relations. The MYCIN system for diagnosing infectious diseases, also developed in the 1970s, uses structured knowledge in the form of production rules, stating that if certain facts are true, then one can conclude certain other facts with a given certainty factor. DXplain, developed starting in the 1980s, uses structured knowledge similar to that of Internist-I, but adds a hierarchical lexicon of findings.

Iliad, developed starting in the 1990s, adds more sophisticated probabilistic reasoning where each disease has an associated a priori probability of the disease (in the population for which Iliad was designed), and a list of findings along with the fraction of patients with the disease who have the finding (sensitivity), and the fraction of patients without the disease who have the finding (1-specificity).

In 2000, diagnosis systems using unstructured knowledge started to appear. These systems use some structuring of knowledge such as, for example, entities such as findings and disorders being tagged in documents to facilitate retrieval. ISABEL, for example, uses Autonomy information retrieval software and a database of medical textbooks to retrieve appropriate diagnoses given input findings. Autonomy Auminence uses the Autonomy technology to retrieve diagnoses given findings and organizes the diagnoses by body system. First CONSULT allows one to search a large collection of medical books, journals, and guidelines by chief complaints and age group to arrive at possible diagnoses. PEPID DDX is a diagnosis generator based on PEPID's independent clinical content.

Clinical decision rules have been developed for a number of medical disorders, and computer systems have been developed to help practitioners and patients apply these rules. The Acute Cardiac Ischemia Time-Insensitive Predictive instrument (ACI-TIPI) takes clinical and ECG features as input and produces probability of acute cardiac ischemia as output to assist with triage of patients with chest pain or other symptoms suggestive of acute cardiac ischemia. ACI-TIPI is incorporated into many commercial heart monitors/defibrillators. The CaseWalker system uses a four-item questionnaire to diagnose major depressive disorder. The PKC Advisor provides guidance on 98 patient problems such as abdominal pain and vomiting.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method, in a data processing system, comprising at least one processor and at least one memory, the at least one memory comprising instructions executed by the at least one processor to cause the at least one processor to implement a cognitive medical treatment recommendation system, is provided for presenting contextually relevant patient data to a medical professional. The illustrative embodiment analyzes electronic medical records (EMRs) of a patient to identify a medical condition associated with the patient. The illustrative embodiment identifies a set of treatments from a corpus of medical treatment guidelines for the patient's medical condition. The illustrative embodiment identifies for each treatment in the set of treatments a subset of treatments that the patient has followed. The illustrative embodiment presents the set of treatments for the patient's medical condition that the patient has followed.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will he described in, or will become apparent to those of ordinary skill in the art in view of, the following detailed description of the example embodiments of the present invention.

### BRIEF DESCRIPTION OF THE SEVERAL VIEWS OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive system in a computer network;

FIG. 2 is a block diagram of an example data processing system in which aspects of the illustrative embodiments are implemented;

FIG. 3 is an example diagram illustrating an interaction of elements of a healthcare cognitive system in accordance with one illustrative embodiment;

FIG. 4 depicts an exemplary flowchart of the operation performed by a cognitive medical treatment recommendation system in presenting contextually relevant patient data to a medical professional in a graphical user interface in accordance with an illustrative embodiment;

FIG. 5 depicts an exemplary flowchart of the operation performed by a cognitive medical treatment recommendation system in determining whether a treatment that the patient has followed has controlled or failed to control the patient's medical condition in accordance with an illustrative embodiment; and

FIG. 6 depicts an additional exemplary flowchart of the operation performed by a cognitive medical treatment recommendation system in presenting contextually relevant patient data to a medical professional in a graphical user interface in accordance with an illustrative embodiment.

### DETAILED DESCRIPTION

The strengths of current cognitive systems, such as current medical diagnosis, patient health management, patient treatment recommendation systems, law enforcement investigation systems, and other decision support systems, are that they can provide insights that improve the decision making performed by human beings. For example, in the medical context, such cognitive systems may improve medical practitioners' diagnostic hypotheses, can help medical practitioners avoid missing important diagnoses, and can assist medical practitioners with determining appropriate treatments for specific diseases. However, current systems still suffer from significant drawbacks which should be addressed in order to make such systems more accurate and usable for a variety of applications as well as more representative of the way in which human beings make decisions, such as diagnosing and treating patients. In particular, one drawback of current systems is that medical professionals have to sift through voluminous amounts of data and documentation in order to identify relevant information pertinent to the treatment of a patient.

In order for the medical professional to be able to have quick and easy access to the most relevant information pertinent to the treatment of a patient, the illustrative embodiments provide mechanisms for presenting contextually relevant patient data to a medical professional in a graphical user interface. The cognitive medical treatment recommendation system generates a graphical user interface (GUI) that is configured to present the most relevant information for treating a patient based on a context of the patient being treated. The cognitive medical treatment recommendation system provides a historical data representation in the GUI of patients that follow each treatment path for a medical condition. The cognitive medical treatment recommendation system also provides information about how many of those patients have their medical condition under control as well as those patients that do not have their medical condition under control. Each row of information presented in the GUI indicates separate treatments, as determined from medical treatment guidelines, with blocks within each row being representative of different medications included in that treatment.

For a particular patient, issues associated with the depicted treatments may be identified and corresponding alerts or notifications may be provided in association with the representation in the GUI. For example, if patient George has a Sulfa allergy and is not able to take a particular drug, a corresponding alert or notification may be associated with treatment rows and blocks in rows where the Sulfa allergy may be a factor. As another example, assume that patient George has already tried a particular medication regimen in other treatments associated with the medical condition and the medication has caused an unwanted side effect or has not improved the medical condition, as indicated in George's electronic medical records (EMRs). The cognitive medical treatment recommendation system may present this information as an alert or notification in association with treatments that have that particular medication regimen as part of the treatment for the medical condition.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

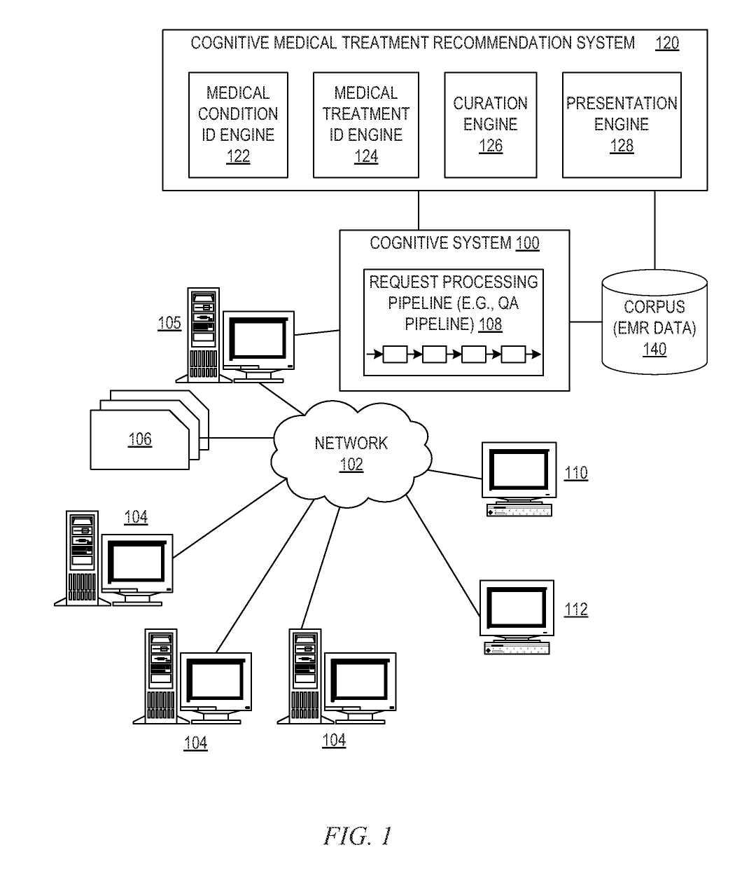

As noted above, the present invention provides mechanisms for presenting contextually relevant patient data to a medical professional in a graphical user interface. Thus, the illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for presenting contextually relevant patient data to a medical professional in a graphical user interface that which implements a request processing pipeline, such as a Question Answering (QA) pipeline (also referred to as a Question/Answer pipeline or Question and Answer pipeline) for example, request processing methodology, and request processing computer program product with which the mechanisms of the illustrative embodiments are implemented. These requests may be provided as structured or unstructured request messages, natural language questions, or any other suitable format for requesting an operation to be performed by the cognitive system. As described in more detail hereafter, the particular application that is implemented in the cognitive system of the present invention is an application for presenting contextually relevant patient data to a medical professional in a graphical user interface.

It should be appreciated that the cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests (or questions in implementations using a QA pipeline), depending on the desired implementation. For example, in some cases, a first request processing pipeline may be trained to operate on input requests directed to a providing a historical data representation in the GUI of patients that follow each treatment path for a medical condition. In other cases, for example, the request processing pipelines may be configured to provide different types of cognitive functions or support different types of applications, such as one request processing pipeline being used for identifying how many patients with a particular medical condition have their medical condition under control as well as patients with the same particular medical condition that do not have their medical condition under control.

Moreover, each request processing pipeline may have its own associated corpus or corpora that they ingest and operate on, e.g., one corpus for medical treatment guideline documents and another corpus for electronic medical record documents in the above examples. In some cases, the request processing pipelines may each operate on the same domain of input questions but may have different configurations, e.g., different annotators or differently trained annotators, such that different analysis and potential answers are generated. The cognitive system may provide additional logic for routing input questions to the appropriate request processing pipeline, such as based on a determined domain of the input request, combining and evaluating final results generated by the processing performed by multiple request processing pipelines, and other control and interaction logic that facilitates the utilization of multiple request processing pipelines.

As noted above, one type of request processing pipeline with which the mechanisms of the illustrative embodiments may be utilized is a Question Answering (QA) pipeline. The description of example embodiments of the present invention hereafter will utilize a QA pipeline as an example of a request processing pipeline that may be augmented to include mechanisms in accordance with one or more illustrative embodiments. It should be appreciated that while the present invention will be described in the context of the cognitive system implementing one or more QA pipelines that operate on an input question, the illustrative embodiments are not limited to such. Rather, the mechanisms of the illustrative embodiments may operate on requests that are not posed as “questions” but are formatted as requests for the cognitive system to perform cognitive operations on a specified set of input data using the associated corpus or corpora and the specific configuration information used to configure the cognitive system. For example, rather than asking a natural language question of “What medical treatments apply to patient P?”, the cognitive system may instead receive a request of “generate a list of medical treatments for the medical condition of patient P,” or the like. It should be appreciated that the mechanisms of the QA system pipeline may operate on requests in a similar manner to that of input natural language questions with minor modifications. In fact, in some cases, a request may be converted to a natural language question for processing by the QA system pipelines if desired for the particular implementation.

As will be discussed in greater detail hereafter, the illustrative embodiments may be integrated in, augment, and extend the functionality of these QA pipeline, or request processing pipeline, mechanisms of a healthcare cognitive system with regard to identifying issues associated with the medical treatments for the medical condition of the patent and presenting a corresponding alerts or notifications in association with the representation in the GUI. For example, identifying one or more medical treatments that include a medication that the patient experienced an adverse reaction to, a medication that has caused unwanted side effects, a medication that has not helped control the patient's medical condition, or the like.

Thus, it is important to first have an understanding of how cognitive systems and question and answer creation in a cognitive system implementing a QA pipeline is implemented before describing how the mechanisms of the illustrative embodiments are integrated in and augment such cognitive systems and request processing pipeline, or QA pipeline, mechanisms. It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

As an overview, a cognitive system is a specialized computer system, or set of computer systems, configured with hardware and/or software logic (in combination with hardware logic upon which the software executes) to enhance human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that enable people and machines to interact in a more natural manner so as to extend and magnify human expertise and cognition. A cognitive system comprises complex logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation., e,g., items of interest to a particular user, potential new contact recommendations, or the like.

IBM Watson™ is an example of one such cognitive system which can process human readable language and identify inferences between text passages with high accuracy at speeds far faster than human beings and on a larger scale. In general, such cognitive systems are able to perform the following functions:
Operate within the pre-determined bounds of human language and understanding,
Ingest and process vast amounts of structured and unstructured data,
Generate and evaluate hypothesis,
Weigh and evaluate responses that are based only on relevant evidence,
Provide situation-specific advice, insights, and guidance,
Enable decision making at the point of impact (contextual guidance),
Scale in proportion to the task,
Extend and magnify human expertise and cognition,
Identify resonating, human-like attributes and traits from natural language,
Deduce various language specific or agnostic attributes from natural language,
High degree of relevant recollection from data points (images, text, voice) (memorization and recall),
Predict and sense with situational awareness that mimic human cognition based on experiences, or
Answer questions based on natural language and specific evidence.

In one aspect, cognitive systems provide mechanisms for answering questions posed to these cognitive systems using a Question Answering pipeline or system (QA system) and/or process requests which may or may not be posed as natural language questions. The QA pipeline or system is a complex application executing on data processing hardware that answers questions pertaining to a given subject-matter domain presented in natural language. The QA pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the QA pipeline. The document may include any file, text, article, or source of data for use in the QA system. For example, a QA pipeline accesses a body of knowledge about the domain, or subject matter area, e,g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users input questions to cognitive system which implements the QA pipeline. The QA pipeline then answers the input questions using the content in the corpus of data by evaluating documents, sections of documents, portions of data in the corpus, or the like. When a process evaluates a given section of a document for semantic content, the process can use a variety of conventions to query such document from the QA pipeline, e.g., sending the query to the QA pipeline as a well-formed question which is then interpreted by the QA pipeline and a response is provided containing one or more answers to the question. Semantic content is content based on the relation between signifiers, such as words, phrases, signs, and symbols, and what they stand for, their denotation, or connotation. In other words, semantic content is content that interprets an expression, such as by using Natural Language Processing.

As will be described in greater detail hereafter, the QA pipeline receives an input question, parses the question to extract the major features of the question, uses the extracted features to formulate queries, and then applies those queries to the corpus of data. Based on the application of the queries to the corpus of data, the QA pipeline generates a set of hypotheses, or candidate answers to the input question, by looking across the corpus of data for portions of the corpus of data that have some potential for containing a valuable response to the input question. The QA pipeline then performs deep analysis on the language of the input question and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the input question and the found portions of the corpus of data. Other reasoning algorithms may look at temporal or spatial features in the language, while others may evaluate the source of the portion of the corpus of data and evaluate its veracity.

The scores obtained from the various reasoning algorithms indicate the extent to which the potential response is inferred by the input question based on the specific area of focus of that reasoning algorithm. Each resulting score is then weighted against a statistical model. The statistical model captures how well the reasoning algorithm performed at establishing the inference between two similar passages for a particular domain during the training period of the QA pipeline. The statistical model is used to summarize a level of confidence that the QA pipeline has regarding the evidence that the potential response, i.e. candidate answer, is inferred by the question. This process is repeated for each of the candidate answers until the QA pipeline identifies candidate answers that surface as being significantly stronger than others and thus, generates a final answer, or ranked set of answers, for the input question.

As mentioned above, QA pipeline mechanisms operate by accessing information from a corpus of data or information (also referred to as a corpus of content), analyzing it, and then generating answer results based on the analysis of this data. Accessing information from a corpus of data typically includes: a database query that answers questions about what is in a collection of structured records, and a search that delivers a collection of document links in response to a query against a collection of unstructured data (text, markup language, etc.). Conventional question answering systems are capable of generating answers based on the corpus of data and the input question, verifying answers to a collection of questions for the corpus of data, correcting errors in digital text using a corpus of data, and selecting answers to questions from a pool of potential answers, i.e. candidate answers.

Content creators, such as article authors, electronic document creators, web page authors, document database creators, and the like, determine use cases for products, solutions, and services described in such content before writing their content. Consequently, the content creators know what questions the content is intended to answer in a particular topic addressed by the content. Categorizing the questions, such as in terms of roles, types of information, tasks, or the like, associated with the question, in each document of a corpus of data allows the QA pipeline to more quickly and efficiently identify documents containing content related to a specific query. The content may also answer other questions that the content creator did not contemplate that may be useful to content users. The questions and answers may be verified by the content creator to be contained in the content for a given document. These capabilities contribute to improved accuracy, system performance, machine learning, and confidence of the QA pipeline. Content creators, automated tools, or the like, annotate or otherwise generate metadata for providing information useable by the QA pipeline to identify these questions and answer attributes of the content.

Operating on such content, the QA pipeline generates answers for input questions using a plurality of intensive analysis mechanisms which evaluate the content to identify the most probable answers, i.e. candidate answers, for the input question. The most probable answers are output as a ranked listing of candidate answers ranked according to their relative scores or confidence measures calculated during evaluation of the candidate answers, as a single final answer having a highest ranking score or confidence measure, or which is a best match to the input question, or a combination of ranked listing and final answer.

FIG. 1 depicts a schematic diagram of one illustrative embodiment of a cognitive system 100 implementing a request processing pipeline 108, which in some embodiments may be a question answering (QA) pipeline, in a computer network 102. For purposes of the present description, it will be assumed that the request processing pipeline 108 is implemented as a QA pipeline that operates on structured and/or unstructured requests in the form of input questions. One example of a question processing operation which may be used in conjunction with the principles described herein is described in U.S. Patent Application Publication No. 2011/0125734, which is herein incorporated by reference in its entirety. The cognitive system 100 is implemented on one or more computing devices 104A-D (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-D. The network 102 includes multiple computing devices 104A-D, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 enables question processing and answer generation (QA) functionality for one or more cognitive system users via their respective computing devices 110-112. In other embodiments, the cognitive system 100 and network 102 may provide other types of cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language question, natural language request for information, natural language request for the performance of a cognitive operation, or the like. For example, the cognitive system 100 receives input from the network 102, a corpus or corpora of electronic documents 140, cognitive system users, and/or other data and other possible sources of input. In one embodiment, some or all of the inputs to the cognitive system 100 are routed through the network 102. The various computing devices 104A-D on the network 102 include access points for content creators and cognitive system users. Some of the computing devices 104A-D includes devices for a database storing the corpus or corpora of data 140 (Which is shown as a separate entity in FIG. 1 for illustrative purposes only). Portions of the corpus or corpora of data 140 may also be provided on one or more other network attached storage devices, in one or more databases, or other computing devices not explicitly shown in FIG. 1. The network 102 includes local network connections and remote connections in various embodiments, such that, the cognitive system 100 may operate in environments of any size, including local and global, e.g., the Internet.

In one embodiment, the content creator creates content in a document of the corpus or corpora of data 140 for use as part of a corpus of data with the cognitive system 100. The document includes any file, text, article, or source of data for use in the cognitive system 100. Cognitive system users access the cognitive system 100 via a network connection or an Internet connection to the network 102, and input questions/requests to the cognitive system 140 that are answered/processed based on the content in the corpus or corpora of data 140. In one embodiment, the questions/requests are formed using natural language. The cognitive system 100 parses and interprets the question/request via a pipeline 108, and provides a response to the cognitive system user, e.g., cognitive system user 110, containing one or more answers to the question posed, response to the request, results of processing the request, or the like. In some embodiments, the cognitive system 100 provides a response to users in a ranked list of candidate answers/responses while in other illustrative embodiments, the cognitive system 100 provides a single final answer/response or a combination of a final answer/response and ranked listing of other candidate answers/responses.

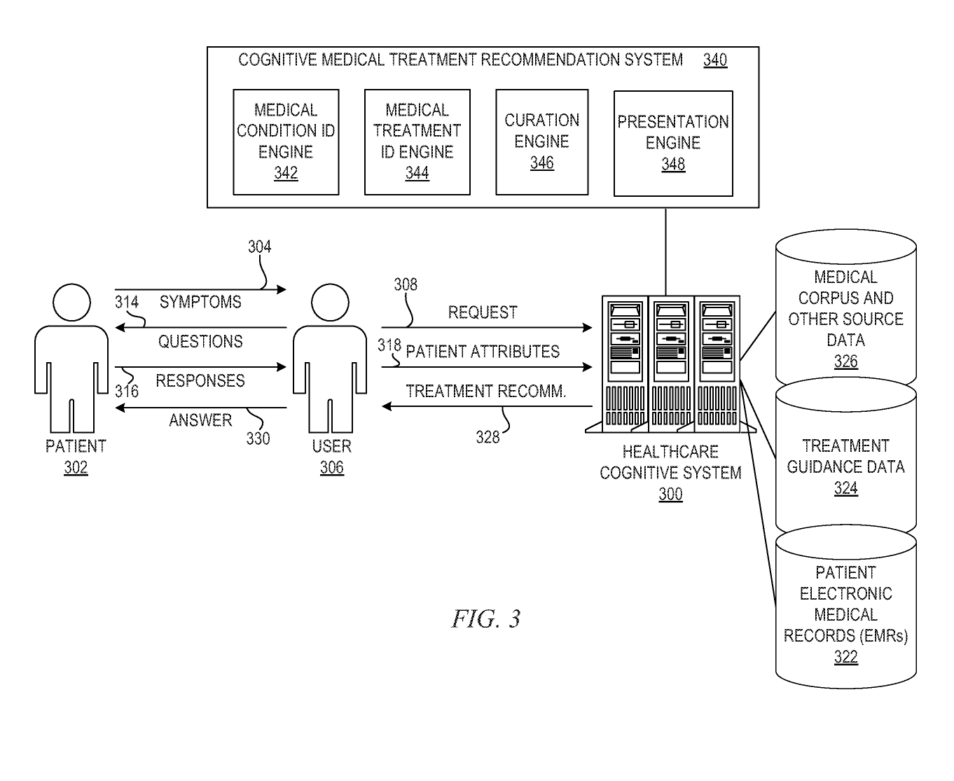

The cognitive system 100 implements the pipeline 108 which comprises a plurality of stages for processing an input question/request based on information obtained from the corpus or corpora of data 140. The pipeline 108 generates answers/responses for the input question or request based on the processing of the input question/request and the corpus or corpora of data 140. The pipeline 108 will he described in greater detail hereafter with regard to FIG. 3.

In some illustrative embodiments, the cognitive system 100 may be the IBM Watson™ cognitive system available from International Business Machines Corporation of Armonk, New York, which is augmented with the mechanisms of the illustrative embodiments described hereafter. As outlined previously, a pipeline of the IBM Watson™ cognitive system receives an input question or request which it then parses to extract the major features of the question/request, which in turn are then used to formulate queries that are applied to the corpus or corpora of data 140. Based on the application of the queries to the corpus or corpora of data 140, a set of hypotheses, or candidate answers/responses to the input question/request, are generated by looking across the corpus or corpora of data 140 for portions of the corpus or corpora of data 140 (hereafter referred to simply as the corpus 140) that have some potential for containing a valuable response to the input question/response (hereafter assumed to be an input question). The pipeline 108 of the IBM Watson™ cognitive system then performs deep analysis on the language of the input question and the language used in each of the portions of the corpus 140 found during the application of the queries using a variety of reasoning algorithms.

The scores obtained from the various reasoning algorithms are then weighted against a statistical model that summarizes a level of confidence that the pipeline 108 of the IBM Watson™ cognitive system 100, in this example, has regarding the evidence that the potential candidate answer is inferred by the question. This process is to be repeated for each of the candidate answers to generate a ranked listing of candidate answers which may then be presented to the user that submitted the input question, e.g., a user of client computing device 110, or from which a final answer is selected and presented to the user. More information about the pipeline 108 of the IBM Watson™ cognitive system 100 may be obtained, for example, from the IBM Corporation website, IBM Redbooks, and the like. For example, information about the pipeline of the IBM Watson™ cognitive system can be found in Yuan et al., “Watson™ and Healthcare,” IBM developerWorks, 2011 and “The Era of Cognitive Systems: An Inside Look at IBM Watson™ and How it Works” by Rob High, IBM Redbooks, 2012.

As noted above, while the input to the cognitive system 100 from a client device may be posed in the form of a natural language question, the illustrative embodiments are not limited to such. Rather, the input question may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for presenting contextually relevant patient data to a medical professional in a graphical user interface. For example, depending upon the particular implementation, the healthcare based operations may comprise patient diagnostics, medical treatment recommendation systems, medical practice management systems, personal patient care plan generation and monitoring, patient electronic medical record (EMR) evaluation for various purposes, such as for identifying patients that are suitable for a medical trial or a particular type of medical treatment, or the like. Thus, the cognitive system 100 may be a healthcare cognitive system 100 that operates in the medical or healthcare type domains and which may process requests for such healthcare operations via the request processing pipeline 108 input as either structured or unstructured requests, natural language input questions, or the like. In one illustrative embodiment, the cognitive system 100 is a cognitive medical treatment recommendation system 100 that analyzes a patient's EMR in relation to medical guidelines and other medical documentation in a corpus of information to generate a recommendation as to how to treat a medical malady or condition of the patient.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing a cognitive medical treatment recommendation system 120 that generates a graphical user interface (GUI) configured to present the most relevant information for treating a patient based on a context of the patient being treated. As shown in FIG. 1, cognitive medical treatment recommendation system 120 comprises medical condition identification engine 122, medical treatment identification engine 124, curation engine 126, and presentation engine 128.

In the initialization of cognitive system 100 and, more specifically, cognitive medical treatment recommendation system 120, medical condition identification engine 122 analyzes electronic medical records (EMR) of a patient stored in corpus or corpora of data 140 to identify a medical condition associated with the patient. As stated previously, corpus or corpora of data 140 may include one corpus for medical treatment guideline documents and another corpus for electronic medical record documents. Thus, in addition to medical condition identification engine 122 analyzing the electronic medical records (EMR) of the patient stored in corpus or corpora of data 140 in order to identify a medical condition associated with the patient, medical treatment identification engine 124 identifies a set of treatments for the patient's medical condition from a set of medical treatment guidelines stored in corpus or corpora of data 140. With the patient's medical condition and the set of treatments for the patient's medical condition identified, curation engine 126 identifies a subset of treatments that the patient has followed as well as, for each treatment in the subset of treatments, whether the treatment has controlled or failed to control the patient's medical condition.

Additionally, for each treatment in the subset of treatments that the patient has followed, curation engine 126 identifies a set of medications utilized in the treatment. Curation engine 126 determines whether the patient experienced an adverse reaction to one or more medications in the set of medications. Responsive to the patient experiencing the adverse reaction to one or more medications in the set of medications, duration engine 126 indicates the one or more medications to which the patient experienced the adverse reaction corresponding to the treatment in the subset of treatments.

Further, for each treatment in the subset of treatments that the patient has followed, curation engine 126 identifies a set of medications utilized in the treatment. Curation engine 126 determines whether the set of medications assisted in controlling the patient's medical condition. The determination may be made based on improved lab results, less symptoms indicated by the patient, or the like. Responsive to the set of medications assisting in controlling the patient's medical condition, curation engine 126 utilizes the determination of the set of medications assisting in controlling the patient's medical condition to provide an identification that the treatment controlled the patient's medical condition. Responsive to the set of medications failing to assist in controlling the patient's medical condition, duration engine 126 utilizes the determination of the set of medications failed to control the patient's medical condition to provide an indication that the treatment failed to control the patient's medical condition.

Still further, for the remainder of the set of treatments for the patient's medical condition that the patient has yet to attempt, curation engine 126 identifies whether any of those treatments would not be feasible for the patient to attempt. That is, based on the requirements associated with each treatment, i.e. medications, physical exercise, weight loss, or the like, one or more of the requirements may rule out the patient even attempting that treatment. Such as, the patient not being able to take a particular medication, not being able to perform a particular physical exercise due to a physical limitation, or the like. Thus, curation engine 126 may provide another indication as to whether, for each treatment in the remainder of treatments for the patient's medical condition that the patient has yet to attempt, the treatment is a feasible to attempt or should not be tried based on the analyzed requirements.

Once the determinations and indications have been provided by curation engine 126, presentation engine 128 presents the set of treatments for the patient's medical condition to a medical professional in a graphical user interface (GUI). The presentation may identify one or more of:
treatments that the patient has followed,
treatments that the patient has followed that controlled the patient's medical condition,
treatments that the patient has followed that failed to control patient's medical condition,
treatments that the patient has followed that failed to control the patient's medical condition and reasons why,
treatments the patient has yet to attempt,
treatments the patient has yet to attempt that are feasible for the patient to attempt,
treatments the patient has yet to attempt that would not be feasible for the patient to attempt, or
treatments the patient has yet to attempt that would not e feasible for the patient to attempt and reasons why.

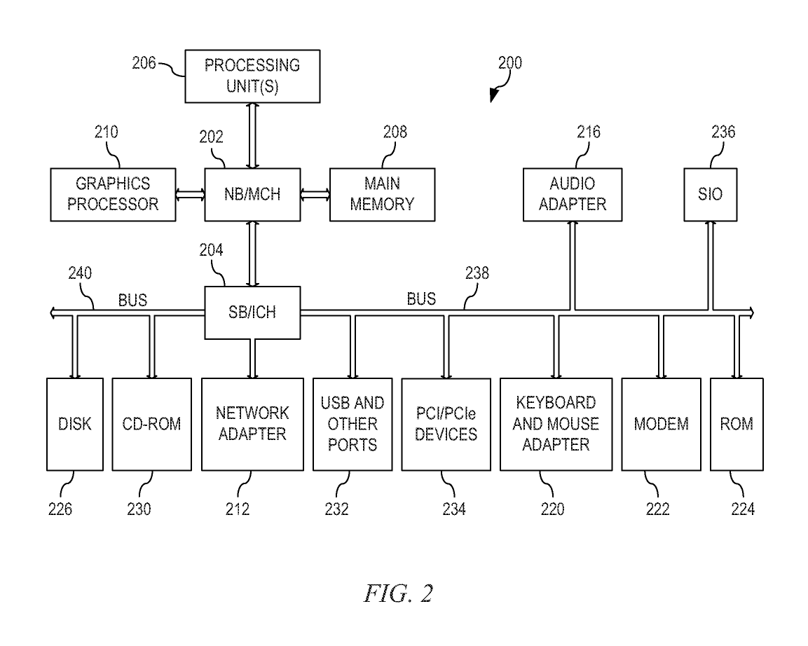

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and QA system pipeline 108 augmented to include the additional mechanisms of the illustrative embodiments described hereafter.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows An object-oriented programming system, such as the Java™ programming system, may run in conjunction with the operating system and provides calls to the operating system from Java™ programs or applications executing on data processing system 200.

As a server, data processing system 200 may be, for example, an IBM® eServer™ System p® computer system, running the Advanced interactive Executive (AIX) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

FIG. 3 is an example diagram illustrating an interaction of elements of a healthcare cognitive system in accordance with one illustrative embodiment. The example diagram of FIG. 3 depicts an implementation of a healthcare cognitive system 300, which may be a cognitive medical treatment recommendation system such as cognitive medical treatment recommendation system 100 described in FIG. 1, that is configured to present contextually relevant patient data to a medical professional in a graphical user interface. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts patient 302 and medical professional 306 as human figures, the interactions with and between these entities may be performed using computing devices, medical equipment, and/or the like, such that entities 302 and 306 may in fact be computing devices, e.g., client computing devices. For example, interactions 304, 314, 316, and 330 between patient 302 and user 306 may be performed orally, e.g., a doctor interviewing a patient, and may involve the use of one or more medical instruments, monitoring devices, or the like, to collect information that may be input to the healthcare cognitive system 300 as patient attributes 318. Interactions between user 306 and healthcare cognitive system 300 will be electronic via a user computing device (not shown), such as a client computing device 110 or 112 in FIG. 1, communicating with healthcare cognitive system 300 via one or more data communication links and potentially one or more data networks.

As shown in FIG. 3, in accordance with one illustrative embodiment, a patient 302 presents symptoms 304 of a medical malady or condition to a user 306, such as a healthcare practitioner, technician, or the like. User 306 may interact with patient 302 via a question 314 and response 316 exchange where user 306 gathers more information about patient 302, symptoms 304, and the medical malady or condition of patient 302. It should be appreciated that the questions/responses may in fact also represent user 306 gathering information from patient 302 using various medical equipment, blood pressure monitors, thermometers, wearable health and activity monitoring devices associated with patient 302 such as a FitBit™, a wearable heart monitor, or any other medical equipment that may monitor one or more medical characteristics of patient 302. In some cases such medical equipment may be medical equipment typically used in hospitals or medical centers to monitor vital signs and medical conditions of patients that are present in hospital beds for observation or medical treatment.

In response, user 306 submits request 308 to healthcare cognitive system 300, such as via a user interface on a client computing device that is configured to allow users to submit requests to healthcare cognitive system 300 in a format that healthcare cognitive system 300 is able to parse and process. Request 308 may include, or be accompanied with, information identifying patient attributes 318. These patient attributes 318 may include, for example, an identifier of patient 302 from which patient EMRs 322 for patient 302 may be retrieved, demographic information about patient 302, symptoms 304, and other pertinent information obtained from responses 316 to questions 314 or information obtained from medical equipment used to monitor or gather data about the condition of patient 302, including a medical conditions associated with patient 302. Any information about patient 302 that may be relevant to a cognitive evaluation of patient 302 by healthcare cognitive system 300 may be included in request 308 and/or patient attributes 318.

Healthcare cognitive system 300 provides a cognitive system that is specifically configured to perform an implementation specific healthcare oriented cognitive operation. In the depicted example, this cognitive medical treatment recommendation operation is directed to providing a set of treatments 328 associated with the medical condition of patient 302 to user 306 to assist user 306 in treating patient 302 based on their reported symptoms 304 and other information gathered about patient 302 via question 314 and response 316 process and/or medical equipment monitoring/data gathering. Healthcare cognitive system 300 operates on request 308 and patient attributes 318 utilizing information gathered from medical corpus and other source data 326, treatment guidance data 324, and patient EMRs 322 associated with the patient 302 to generate one or more treatment recommendation 328. Treatment recommendations 328 may be presented with associated supporting evidence, obtained from the patient attributes 318 and data sources 322, 324, and 326, indicating the reasoning as to why the treatment recommendation 328 is being provided.

For example, based on request 308 and patient attributes 318, healthcare cognitive system 300 may operate on the request to parse request 308 and patient attributes 318 to determine what is being requested and the criteria upon which the request is to be generated as identified by patient attributes 318, and may perform various operations for generating queries that are sent to the data sources 322, 324, and 326 to retrieve data, generate associated indications associated with the data, and provides supporting evidence found in the data sources 322, 324, and 326. In the depicted example, patient EMRs 322 is a patient information repository that collects patient data from a variety of sources, e.g., hospitals, laboratories, physicians' offices, health insurance companies, pharmacies, etc. Patient EMRs 322 store various information about individual patients, such as patient 302, in a manner (structured, unstructured, or a mix of structured and unstructured formats) that the information may be retrieved and processed by healthcare cognitive system 300. This patient information may comprise various demographic information about patients, personal contact information about patients, employment information, health insurance information, laboratory reports, physician reports from office visits, hospital charts, historical information regarding previous diagnoses, symptoms, treatments, prescription information, etc. Based on an identifier of the patient 302, the patient's corresponding EMRs 322 from this patient repository may be retrieved by healthcare cognitive system 300 and searched/processed to generate treatment recommendations 328.

Treatment guidance data 324 provides a knowledge base of medical knowledge that is used to identify potential treatments for a patient's medical condition based on patient's attributes 318 and historical information presented in patient's EMRs 322. Treatment guidance data 324 may be obtained from official treatment guidelines and policies issued by medical authorities, e.g., the American Medical Association, may be obtained from widely accepted physician medical and reference texts, e.g., the Physician's Desk Reference, insurance company guidelines, or the like. The treatment guidance data 324 may be provided in any suitable form that may be ingested by the healthcare cognitive system 300 including both structured and unstructured formats.

In some cases, such treatment guidance data 324 may be provided in the form of rules that indicate the criteria required to be present, and/or required not to be present, for the corresponding treatment to be applicable to a particular patient for treating a particular symptom or medical malady/condition. For example, the treatment guidance data 324 may comprise a treatment recommendation rule that indicates that for a treatment of Decitabine, strict criteria for the use of such a treatment is that patient 302 is less than or equal to 60 years of age, has acute myeloid leukemia (AML), and no evidence of cardiac disease. Thus, for a patient 302 that is 59 years of age, has AML, and does not have any evidence in their patient attributes 318 or patient EMRs 322 indicating evidence of cardiac disease, the following conditions of the treatment rule exist:

Age<=60 years=59 (MET);

Patient has AML=AML (MET); and

Cardiac Disease=false (MET)

Since all of the criteria of the treatment rule are met by the specific information about this patient 302, then the treatment of Decitabine is a candidate treatment recommendation for consideration for this patient 302. However, if the patient had been 69 years old, the first criterion would not have been met and the Decitabine treatment would not be a candidate treatment recommendation for consideration for this patient 302. Various potential treatment recommendations may be evaluated by healthcare cognitive system 300 based on ingested treatment guidance data 324 to identify subsets of candidate treatment recommendations for further consideration by healthcare cognitive system 300 by identifying such candidate treatment recommendations based on evidential data obtained from patient EMRs 322 and medical corpus and other source data 326.

For example, data mining processes may be employed to mine the data in sources 322 and 326 to identify evidential data supporting and/or refuting the applicability of the candidate treatment recommendations to the particular patient 302 as characterized by the patient's patient attributes 318 and EMRs 322. For example, for each of the criteria of the treatment rule, the results of the data mining provides a set of evidence that supports giving the treatment in the cases where the criterion is “MET” and in cases where the criterion is “NOT MET.” Healthcare cognitive system 300 processes the evidence in accordance with various cognitive logic algorithms to generate an indicator for each candidate treatment recommendation indicating a confidence that the corresponding candidate treatment recommendation is valid for patient 302. The candidate treatment recommendations may then be presented to user 306 as a listing of treatment recommendations 328. Treatment recommendations 328 may be presented to user 306 in a manner that the underlying evidence evaluated by healthcare cognitive system 300 may be accessible, such as via a drilldown interface, so that user 306 may identify the reasons why treatment recommendation 328 is being provided by healthcare cognitive system 300.

In accordance with the illustrative embodiments herein, healthcare cognitive system 300 is augmented to include cognitive medical treatment recommendation system 340. Cognitive medical treatment recommendation system 340 comprises medical condition identification engine 342, medical treatment identification engine 344, curation engine 346, and presentation engine 348 which operate in a similar manner as previously described above with regard to corresponding elements 122-128 in FIG. 1. That is, medical condition identification engine 342 initially analyzes electronic medical records (EMR) of patient 302 stored in patient EMR corpus 322 to identify a medical condition associated with patient 302. Medical treatment identification engine 344 then identifies a set of treatments for the patient's medical condition from a set of medical treatment guidelines stored in treatment guidance data 324 and/or medical corpus and other source data 326. With the patient's medical condition and the set of treatments for the patient's medical condition identified, curation engine 346 identifies a subset of treatments that patient 302 has followed as well as, for each treatment in the subset of treatments, whether the treatment has controlled or failed to control the patient's medical condition.

Additionally, for each treatment in the subset of treatments that patient 302 has followed, curation engine 346 identifies a set of medications utilized in the treatment. Curation engine 346 determines whether patient 302 experienced an adverse reaction to one or more medications in the set of medications. Responsive to patient 302 experiencing the adverse reaction to one or more medications in the set of medications, curation engine 346 indicates the one or more medications to which patient 302 experienced the adverse reaction corresponding to the treatment in the subset of treatments.

Further, for each treatment in the subset of treatments that patient 302 has followed, curation engine 346 identifies a set of medications utilized in the treatment. Curation engine 346 determines whether the set of medications assisted in controlling the patient's medical condition. The determination may be made based on improved lab results, less symptoms indicated by the patient, or the like. Responsive to the set of medications assisting in controlling the patient's medical condition, curation engine 346 utilizes the determination of the set of medications assisting in controlling the patient's medical condition to provide an identification that the treatment controlled the patient's medical condition. Responsive to the set of medications failing to control the patient's medical condition, curation engine 346 utilizes the determination of the set of medications failed to assist in controlling the patient's medical condition to provide an indication that the treatment failed to control the patient's medical condition.

Still further, for the remainder of the set of treatments for the patient's medical condition that patient 302 has yet to attempt, curation engine 346 identifies whether any of those treatments would not be feasible for patient 302 to attempt. That is, based on the requirements associated with each treatment, i.e. medications, physical exercise, weight loss, or the like, one or more of the requirements may rule out patient 302 even attempting that treatment. Such as, patient 302 not being able to take a particular medication, not being able to perform a particular physical exercise due to a physical limitation, or the like. Thus, curation engine 346 may provide another indication as to whether, for each treatment in the remainder of treatments for the patient's medical condition that the patient has yet to attempt, the treatment is a feasible to attempt or should not be tried based on the analyzed requirements.

Once the determinations and indications have been provided by curation engine 346, presentation engine 348 presents the set of treatments for the patient's medical condition to a medical professional in a graphical user interface (GUI). The presentation may identify one or more of:
treatments that the patient has followed,
treatments that the patient has followed that controlled the patient's medical condition,
treatments that the patient has followed that failed to control the patient's medical condition,
treatments that the patient has followed that failed to control the patient's medical condition and reasons why,
treatments the patient has yet to attempt,
treatments the patient has yet to attempt that are feasible for the patient to attempt,
treatments the patient has yet to attempt that would not be feasible for the patient to attempt, or
treatments the patient has yet to attempt that would not feasible for the patient to attempt and reasons why.

Thus, the illustrative embodiments provide mechanisms for presenting contextually relevant patient data to a medical professional in a graphical user interface. The cognitive medical treatment recommendation system generates a graphical user interface (GUI) that is configured to present the most relevant information for treating a patient based on a context of the patient being treated. The cognitive medical treatment recommendation system provides a historical data representation in the GUI of patients that follow each treatment path for a medical condition. The cognitive medical treatment recommendation system also provides information about how many of those patients have their medical condition under control as well as those patients that do not have their medical condition under control. Each row of information presented in the GUI indicates separate treatments, as determined from medical treatment guidelines, with blocks within each row being representative of different medications included in that treatment.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

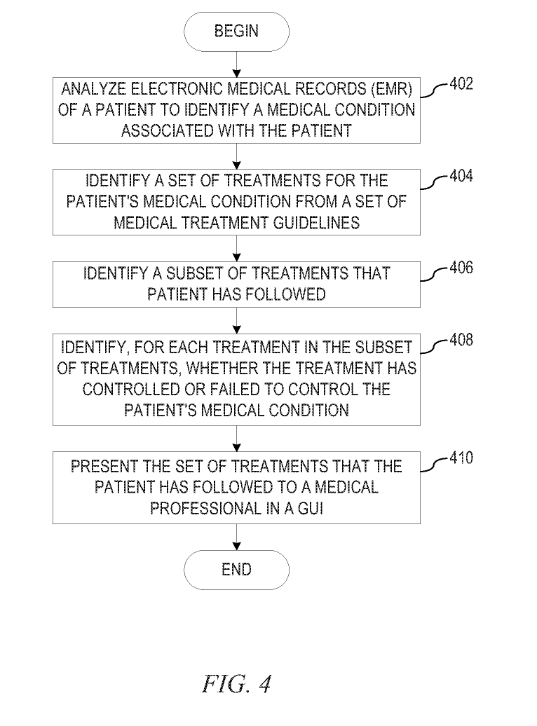

FIG. 4 depicts an exemplary flowchart of the operation performed by a cognitive medical treatment recommendation system in presenting contextually relevant patient data to a medical professional in a graphical user interface in accordance with an illustrative embodiment. As the operation begins, the cognitive medical treatment recommendation system analyzes electronic medical records (EMR) of a patient to identify a medical condition associated with the patient (step 402). The cognitive medical treatment recommendation system then identifies a set of treatments for the patient's medical condition from a set of medical treatment guidelines (step 404). With the patient's medical condition and the set of treatments for the patient's medical condition identified, the cognitive medical treatment recommendation system identifies a subset of treatments that patient has followed (step 406). The cognitive medical treatment recommendation system also identifies, for each treatment in the subset of treatments, whether the treatment has controlled or failed to control the patient's medical condition (step 408). The cognitive medical treatment recommendation system then presents the set of treatments that the patient has followed to a medical professional in a graphical user interface (GUI) (step 410) with an identification of one or more of:
treatments that the patient has followed,
treatments that the patient has followed that controlled the patient's medical condition,
treatments that the patient has followed that failed to control the patient's medical condition, or
treatments that the patient has followed that failed to control the patient's medical condition and reasons why.
   After presenting the set of treatments that the patient has followed to a medical professional in the graphical user interface, the operation terminates.

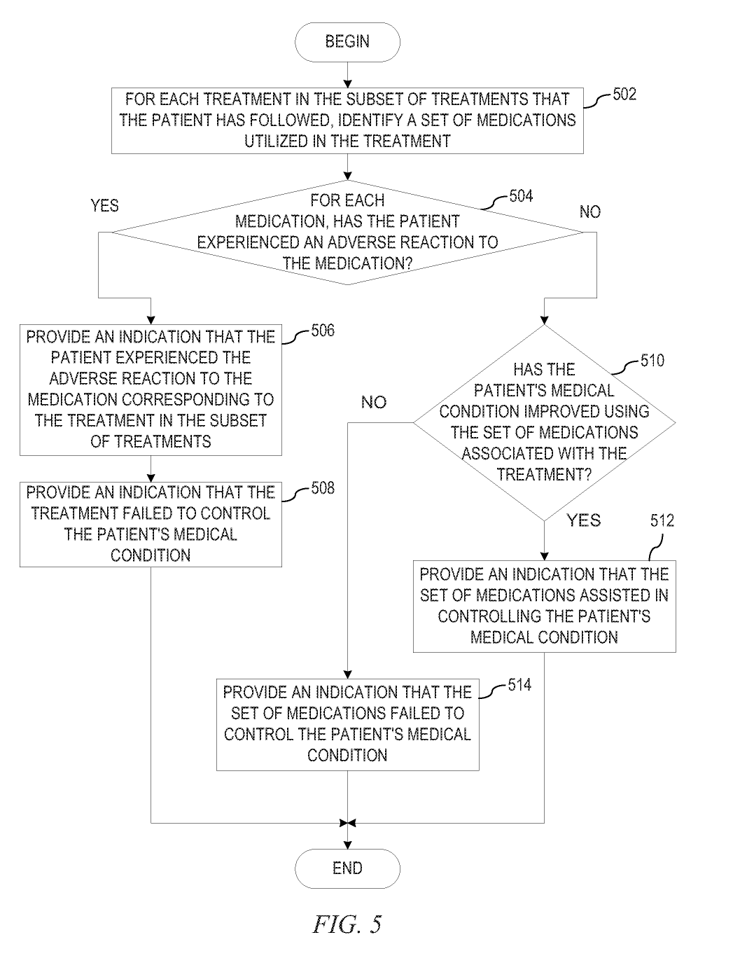

FIG. 5 depicts an exemplary flowchart of the operation performed by a cognitive medical treatment recommendation system in determining whether a treatment that the patient has followed has controlled or failed to control the patient's medical condition in accordance with an illustrative embodiment. For each treatment in the subset of treatments that the patient has followed, the cognitive medical treatment recommendation system identifies a set of medications utilized in the treatment (step 502). For each medication in the set of medications, the cognitive medical treatment recommendation system determines whether the patient experienced an adverse reaction to the medication (step 504). If at step 504 the cognitive medical treatment recommendation system determines that the patient experienced an adverse reaction to the medication, the cognitive medical treatment recommendation system provides an indication that the patient experienced the adverse reaction to the medication corresponding to the treatment in the subset of treatments (step 506) and provides an indication that the treatment failed to control the patient's medical condition (step 508), with the operation ending thereafter.

If at step 504 the cognitive medical treatment recommendation system determines that the patient failed to experience an adverse reaction to the medication, the cognitive medical treatment recommendation system determines whether the patient's medical condition has improved using the set of medications associated with the treatment (step 510). The determination may be made based on improved lab results, less symptoms indicated by the patient, or the like. If at step 510 the cognitive medical treatment recommendation system determines that the patient's medication condition has improved using the set of medications, the cognitive medical treatment recommendation system provides an indication that the set of medications assisted in controlling the patient's medical condition (step 512), with the operation ending thereafter. If at step 510 the cognitive medical treatment recommendation system determines that the patient's medication condition has not improved using the set of medications, the cognitive medical treatment recommendation system provides an indication that the set of medications failed to control the patient's medical condition (step 514), with the operation ending thereafter.

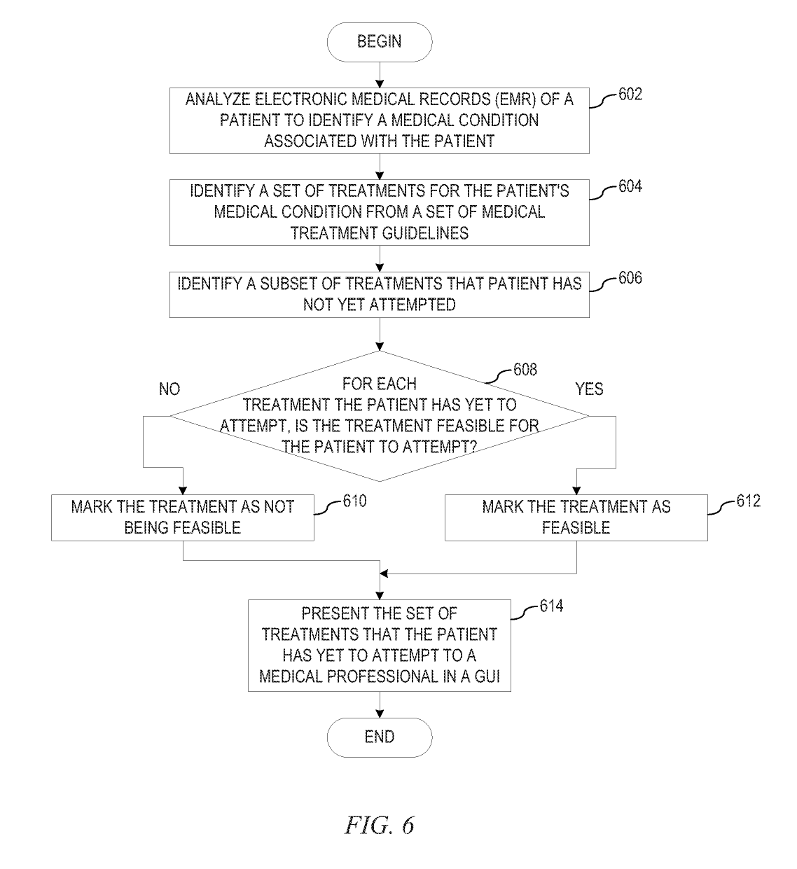

FIG. 6 depicts an additional exemplary flowchart of the operation performed by a cognitive medical treatment recommendation system in presenting contextually relevant patient data to a medical professional in a graphical user interface in accordance with an illustrative embodiment. As the operation begins, the cognitive medical treatment recommendation system analyzes electronic medical records (EMR) of a patient to identify a medical condition associated with the patient (step 602). The cognitive medical treatment recommendation system then identifies a set of treatments for the patient's medical condition from a set of medical treatment guidelines (step 604). With the patient's medical condition and the set of treatments for the patient's medical condition identified, the cognitive medical treatment recommendation system identifies a subset of treatments that patient has not yet attempted (step 606). For each treatment the patient has yet to attempt, the cognitive medical treatment recommendation system determines whether any of those treatments would not be feasible for the patient to attempt (step 608) based on the requirements associated with each treatment, i.e. medications, physical exercise, weight loss, or the like, one or more of the requirements may rule out the patient even attempting that treatment. If at step 608 the cognitive medical treatment recommendation system determines that the treatment is not feasible for the patient to attempt, the cognitive medical treatment recommendation system marks the treatment as not being feasible (step 610) with supporting documentation. If at step 608 the cognitive medical treatment recommendation system determines that the treatment is feasible for the patient to attempt, the cognitive medical treatment recommendation system marks the treatment as feasible (step 612). From steps 610 and 612, the cognitive medical treatment recommendation system presents the set of treatments that the patient has yet to attempt to a medical professional in a graphical user interface (GUI) (step 614) with an identification of one or more of:
treatments the patient has yet to attempt,
   treatments the patient has yet to attempt that are feasible for the patient to attempt,
treatments the patient has yet to attempt that would not be feasible for the patient to attempt, or
treatments the patient has yet to attempt that would not be feasible for the patient to attempt and reasons why.
   After presenting the set of treatments that the patient has yet to attempt to the medical professional in the graphical user interface, the operation terminates.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for presenting contextually relevant patient data to a medical professional in a graphical user interface. The cognitive medical treatment recommendation system generates a graphical user interface (GUI) that is configured to present the most relevant information for treating a patient based on a context of the patient being treated. The cognitive medical treatment recommendation system provides a historical data representation in the GUI of patients that follow each treatment path for a medical condition. The cognitive medical treatment recommendation system also provides information about how many of those patients have their medical condition under control as well as those patients that do not have their medical condition under control. Each row of information presented in the GUI indicates separate treatments, as determined from medical treatment guidelines, with blocks within each row being representative of different medications included in that treatment.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802,11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

1-20. (canceled)

21. A method, in a data processing system comprising at least one processor and at least one memory, the at least one memory comprising instructions executed by the at least one processor to cause the at least one processor to implement a cognitive medical treatment recommendation system, wherein the cognitive medical treatment recommendation system operates to:
analyzing, by the cognitive medical treatment recommendation system, electronic medical records (EMRs) of a patient to identify a medical condition associated with the patient;
identifying, by the cognitive medical treatment recommendation system, a set of treatments from a corpus of medical treatment guidelines for the patient's medical condition;
for each treatment in the set of treatments, identifying, by the cognitive medical treatment recommendation system, a subset of treatments that the patient has followed and a subset of treatments that the patient has yet to attempt; and
presenting, by the cognitive medical treatment recommendation system in a graphical user interface, the set of treatments for the patient's medical condition that the patient has followed and the subset of treatments that the patient has yet to attempt.

22. The method of claim 21, further comprising:
for each treatment of the subset of treatments that the patient has followed, determining, by the cognitive medical treatment recommendation system, whether the treatment in the subset of treatments controlled or failed to control the patient's medical condition; and
presenting, by the cognitive medical treatment recommendation system in the graphical user interface, the set of treatments for the patient's medical condition that the patient has followed that controlled the patient's medical condition.

23. The method of claim 21, further comprising:
for each treatment of the subset of treatments that the patient has followed, determining, by the cognitive medical treatment recommendation system, whether the treatment in the subset of treatments controlled or failed to control the patient's medical condition; and
presenting, by the cognitive medical treatment recommendation system in the graphical user interface, the set of treatments for the patient's medical condition that the patient has followed that failed to control the patient's medical condition.

24. The method of claim 21, further comprising:
for each treatment in the subset of treatments that the patient has followed:
identifying, by the cognitive medical treatment recommendation system, a set of medications utilized in the treatment;
determining, by the cognitive medical treatment recommendation system, whether the patient experienced an adverse reaction to one or more medications in the set of medications; and
responsive to the patient experiencing the adverse reaction to one or more medications in the set of medications, presenting, by the cognitive medical treatment recommendation system in the graphical user interface, an indication of the one or more medications to which the patient experienced the adverse reaction corresponding to the treatment in the subset of treatments that the patient has followed.

25. The method of claim 21, wherein determining whether the treatment in the subset of treatments controlled or failed to control the patient's medical condition further comprises:
for each treatment in the subset of treatments that the patient has followed:
identifying, by the cognitive medical treatment recommendation system, a set of medications utilized in the treatment;
determining, by the cognitive medical treatment recommendation system, whether the patient's medical condition has improved using the set of medications; and
responsive to the patient's medical condition improving, utilizing, by the cognitive medical treatment recommendation system, presenting, by the cognitive medical treatment recommendation system in the graphical user interface, an indication that the set of medications assisted in controlling the patient's medical condition.

26. The method of claim 25, further comprising:
responsive to the patient's medical condition failing to improve, presenting, by the cognitive medical treatment recommendation system in the graphical user interface, an indication that the set of medications failed to assist in controlling the patient's medical condition.

27. The method of claim 21, further comprising:
for each treatment in the subset of treatments that the patient has yet to attempt, determining, by the cognitive medical treatment recommendation system, whether the treatment is feasible for the patient to attempt based on the requirements associated with each treatment;
responsive to the treatment not being feasible for the patient to attempt, marking, by the cognitive medical treatment recommendation system, the treatment as not feasible;
responsive to the treatment being feasible for the patient to attempt, marking, by the cognitive medical treatment recommendation system, the treatment as feasible; and
in presenting the set of treatments for the patient's medical condition that the patient has yet to attempt, identifying, by the cognitive medical treatment recommendation system in the graphical user interface, treatments the patient has yet to attempt that are feasible for the patient to attempt and treatments the patient has yet to attempt that would not be feasible for the patient to attempt.

28. A computer program product comprising a computer readable storage medium having a computer readable program stored therein, wherein the computer readable program, when executed on a computing device, causes the computing device to:
analyze electronic medical records (EMRs) of a patient to identify a medical condition associated with the patient;
identify a set of treatments from a corpus of medical treatment guidelines for the patient's medical condition;
for each treatment in the set of treatments, identify a subset of treatments that the patient has followed and a subset of treatments that the patient has yet to attempt; and
present, in a graphical user interface, the set of treatments for the patient's medical condition that the patient has followed and the subset of treatments that the patient has yet to attempt.

29. The computer program product of claim 28, wherein the computer readable program further causes the computing device to:
for each treatment of the subset of treatments that the patient has followed, determine whether the treatment in the subset of treatments controlled or failed to control the patient's medical condition; and
present, in the graphical user interface, the set of treatments for the patient's medical condition that the patient has followed that controlled the patient's medical condition.

30. The computer program product of claim 28, wherein the computer readable program further causes the computing device to:
for each treatment of the subset of treatments that the patient has followed, determine whether the treatment in the subset of treatments controlled or failed to control the patient's medical condition; and
present, in the graphical user interface, the set of treatments for the patient's medical condition that the patient has followed that failed to control the patient's medical condition.

31. The computer program product of claim 28, wherein the computer readable program further causes the computing device to:
for each treatment in the subset of treatments that the patient has followed:
identify a set of medications utilized in the treatment;
determine whether the patient experienced an adverse reaction to one or more medications in the set of medications; and
responsive to the patient experiencing the adverse reaction to one or more medications in the set of medications, present, in the graphical user interface, an indication of the one or more medications to which the patient experienced the adverse reaction corresponding to the treatment in the subset of treatments that the patient has followed.

32. The computer program product of claim 28, wherein the computer readable program for determining whether the treatment in the subset of treatments controlled or failed to control the patient's medical condition further causes the computing device to:
for each treatment in the subset of treatments that the patient has followed:
identify a set of medications utilized in the treatment;
determine whether the patient's medical condition has improved using the set of medications; and
responsive to the patient's medical condition improving, utilizing, by the cognitive medical treatment recommendation system, present, in the graphical user interface, an indication that the set of medications assisted in controlling the patient's medical condition.

33. The computer program product of claim 32, wherein the computer readable program further causes the computing device to:
responsive to the patient's medical condition failing to improve, providing, by the cognitive medical treatment recommendation system in the graphical user interface, an indication that the set of medications failed to assist in controlling the patient's medical condition.

34. The computer program product of claim 28, wherein the computer readable program further causes the computing device to:
for each treatment in the subset of treatments that the patient has yet to attempt, determine whether the treatment is feasible for the patient to attempt based on the requirements associated with each treatment;
responsive to the treatment not being feasible for the patient to attempt, mark the treatment as not feasible;
responsive to the treatment being feasible for the patient to attempt, mark the treatment as feasible; and
in presenting the set of treatments for the patient's medical condition that the patient has yet to attempt, identify, in the graphical user interface, treatments the patient has yet to attempt that are feasible for the patient to attempt and treatments the patient has yet to attempt that would not be feasible for the patient to attempt.

35. An apparatus comprising:
a processor; and
a memory coupled to the processor, wherein the memory comprises instructions which, when executed by the processor, cause the processor to:
analyze electronic medical records (EMRs) of a patient to identify a medical condition associated with the patient;
identify a set of treatments from a corpus of medical treatment guidelines for the patient's medical condition;
for each treatment in the set of treatments, identify a subset of treatments that the patient has followed and a subset of treatments that the patient has yet to attempt; and
present, in a graphical user interface, the set of treatments for the patient's medical condition that the patient has followed and the subset of treatments that the patient has yet to attempt.

36. The apparatus of claim 35, wherein the instructions further cause the processor to:
for each treatment of the subset of treatments that the patient has followed, determine whether the treatment in the subset of treatments controlled or failed to control the patient's medical condition; and
present, in the graphical user interface, the set of treatments for the patient's medical condition that the patient has followed that controlled the patient's medical condition.

37. The apparatus of claim 35, wherein the instructions further cause the processor to:
for each treatment of the subset of treatments that the patient has followed, determine whether the treatment in the subset of treatments controlled or failed to control the patient's medical condition; and
present, in the graphical user interface, the set of treatments for the patient's medical condition that the patient has followed that failed to control the patient's medical condition.

38. The apparatus of claim 35, wherein the instructions further cause the processor to:
for each treatment in the subset of treatments that the patient has followed:
identify a set of medications utilized in the treatment;
determine whether the patient experienced an adverse reaction to one or more medications in the set of medications; and
responsive to the patient experiencing the adverse reaction to one or more medications in the set of medications, present, in the graphical user interface, an indication of the one or more medications to which the patient experienced the adverse reaction corresponding to the treatment in the subset of treatments that the patient has followed.

39. The apparatus of claim 35, wherein the instructions for determining whether the treatment in the subset of treatments controlled or failed to control the patient's medical condition further cause the processor to:
for each treatment in the subset of treatments that the patient has followed:
identify a set of medications utilized in the treatment;
determine whether the patient's medical condition has improved using the set of medications; and
responsive to the patient's medical condition improving, utilizing, by the cognitive medical treatment recommendation system, present, in the graphical user interface, an indication that the set of medications assisted in controlling the patient's medical condition.

40. The apparatus of claim 39, wherein the instructions further cause the processor to:
responsive to the patient's medical condition failing to improve, providing, by the cognitive medical treatment recommendation system in the graphical user interface, an indication that the set of medications failed to assist in controlling the patient's medical condition.
